(19) United States
(12) Patent Application Publication
(10) Pub. No.: US 2008/0077204 A1
(43) Pub. Date: Mar. 27, 2008

(54) Optical biofilm therapeutic treatment

(75) Inventor: Eric Bornstein, Natick, MA (US)

Correspondence Address: MCDERMOTT WILL & EMERY LLP, 28 STATE STREET, BOSTON, MA, 02109-1775, US

(73) Assignee: Nomir Medical Technologies, Inc., Natick, MA (US)

(21) Appl. No.: 11/880,769

(22) Filed: Jul. 24, 2007

Related U.S. Application Data
(63) Continuation-in-part of application No. 10/961,796, filed on Oct. 8, 2004.
(60) Provisional application No. 60/509,685, filed on Oct. 8, 2003.
(60) Provisional application No. 60/832,770, filed on Jul. 24, 2006.
(60) Provisional application No. 60/832,893, filed on Jul. 24, 2006.

(30) Foreign Application Priority Data
Oct. 8, 2004 (US) PCT/US04/33431

(51) Int. Cl. A61N 5/06

(52) U.S. Cl. 607/100; 607/105; 607/89; 607/93

(57) ABSTRACT

Optical therapeutic treatment devices, systems, apparatus, methods, and techniques are disclosed. Embodiments can include a housing extending along a central axis X, an elongated fiber guide coupled to the housing and adapted to receive an optical fiber having a proximal end and a distal end, a reflector assembly within the housing and extending along the central axis X. The distal end of the optical fiber can includes a carbonized tip within the reflector assembly. The reflector assembly is adapted to reflect the optical energy emitted from the distal end and propagating radially with respect to the central is, so that the reflected optical energy propagates at least in part along a propagation axis parallel to the central axis. Embodiments can utilize free space optics/transmission. Further embodiments can utilize NIR radiation (e.g., including 870 and 930 nm) that is suitable to cause free radical formation in microbes.

### RELATED APPLICATIONS

This application is a continuation-in-part of U.S. application Ser. No. 10/961,796 (as well as PCT Application Serial No. PCT/US2004/033431), filed 8 Oct. 2004, which claims the benefit of U.S. Provisional Application Ser. No. 60/509,685, filed 8 Oct. 2003; the contents of both of which applications are incorporated in their entireties herein by reference; this application further claims the benefit of U.S. Provisional Application Ser. No. 60/832,770 and U.S. Provisional Application Ser. No. 60/832,893, both filed 24 Jul. 2006, both of which application are incorporated in their entireties herein by reference.

### BACKGROUND

The word “biofilm” is often used to describe a community of microorganisms that are enclosed in a mucinous like polymer matrix. Biofilms often consist of many species of bacteria and archaea (and can include fungus), which are typically all held together and protected by a matrix of excreted polymeric compounds. A common biofilm matrix is formed from exopolysaccharide (EPS), water and microbes in percentages of roughly 5% (EPS), 92% (water) and 3% (microbes). The EPS component is an extremely hydrated gel-like (mucinous) bio-polymer that creates a 3-dimensional structure of the biofilm. The EPS matrix protects the microbes within the biofilm from attack by harmful antimicrobial agents (antibiotics) and the immune system of a human body.

Biofilms can be blamed for a myriad of human diseases. For example, dental plaque and subgingival bacterial colonies are living biofilms. Within biofilms, bacteria have the ability to regulate the expression of certain genes in a population-dependent manner, (a phenomenon known as quorum sensing) that allows the higher aggregation of bacteria to become more resistant and dangerous, once the biofilm forms. Because the polymer matrix of a biofilm usually offers resistance to the bacteria from antibiotics, host immune and defense systems, and conventional cleaning agents, biofilms that cause human and animal diseases are typically very difficult to be treated.

One example of biofilm mediated morbidity is seen in individuals with implantable medical devices, for example artificial joints, which are susceptible to biofilm attachment and colonization. In the prior art, treatment for patients with an infected implanted prosthetic joint has consisted of replacement of the implanted artificial joint with a new artificial joint. This not only causes a great difficulty for the patient, but increases the treatment cost of the patient.

Therefore, there is a need for novel systems, apparatus, methods, and techniques for targeting biofilm directly to combat the recalcitrant biofilms, e.g., ones that harbor and protect pathogenic bacteria and/or fungus in tissue or implantable devices.

### SUMMARY OF THE DISCLOSURE

The present disclosure provides an apparatus, systems, methods, and techniques to treat and/or kill biofilms(s) (e.g., including bacteria and fungi) by thermal interaction with the biofilm on/in infected tissue and/or implanted prosthetic device(s) with minimal, if any, harm to the healthy tissue.

According to one preferred embodiment of the present disclosure, an optical therapeutic treatment device includes a housing extending along a central axis X, a guide (e.g., an elongated fiber guide or an black body element guide/holding structure) at least partially received in the housing and adapted to receive an optical fiber having a proximal end and a distal end, a reflector assembly within the housing and extending along the central axis X. The fiber guide is adapted to position a distal end of an optical fiber received therein, to be aligned with the central axis X and within the reflector assembly. The reflector assembly preferably has a parabolic cross-section taken along the central axis X. The reflector assembly defines a central bore for receiving the distal end of the optical fiber. The reflector assembly is adapted to reflect optical energy propagating with a radial component with respect to the central axis X, so that the reflected optical energy propagates at least in part along a propagation axis parallel to the central axis X.

The optical therapeutic device may further include an optical fiber having a distal end portion received within the fiber guide, where the optical fiber includes a carbonized distal end, also referred to herein as a “hot tip”. According to a further embodiment, the optical therapeutic device further includes an energy source and an associated coupling assembly for introducing energy generated by the source to the proximal end of the fiber or black body element. In exemplary embodiments, the energy includes an optical source.

According to one aspect of the present disclosure, the optical energy source is adapted to generate near-infrared (NIR) energy. The optical energy may be coherent (i.e., from a laser) or non-coherent, such as by diode or superluminous diode, etc. In other embodiments, the energy source used to create incandescent secondary emission can include electrical sources, free electron lasers, or other energy sources suitable to cause the black body element to radiate incandescent radiation.

In addition to being a primary emission delivery device for laser photons to a target tissue, laser delivery fibers (and handpiece) can act as “Hot Tip” cutting devices. During a procedure, when an unclad fiber tip is pre-initiated, or comes in contact with tissue, biological matter, biofilm and or blood, and the laser is turned on, the tip will immediately carbonize. This carbonization will instantaneously absorb the intense infrared laser energy propagating through the fiber, which will cause the tip to further heat up and become red hot (above about 726 Centigrade). Once this occurs, the tip of the fiber will in effect, become what is known as a “Black Body Radiator” that generates a secondary visible optical emission, as it becomes incandescent and glows. As progressively more photons from the laser continue to bombard the black carbonized tip of the fiber, the temperature rise to this carbonized black tip is profound and rapid. It is this intense heat of the carbonized (glowing) fiber tip that is known as the “hot tip” for diode laser contact-cutting procedures. Embodiments of the present disclosure make use of one or more of such “hot tips” in novels ways/implementations for purposes of treating/mitigating undesirable biological contaminants such as biofilms, by focusing and transmitting this secondary blackbody energy in a forward direction.

In embodiments of the present disclosure, an optical fiber (supplied with energy from a source such as a light-emitting diode or diode laser) can be placed in a parabolic (or other) handpiece and “pre-initiated” to form a “hot tip”. With the “hot tip” the photobiology and laser-tissue thermodynamics of the interactions in the biofilm and tissue are profoundly different from those found when using a non-carbonized fiber that emits only the primary emission (single wavelength) near-infrared photons at its distal end. When emitted photons from a “Hot Tip” laser fiber are directed (in a parabolic (or other) handpiece) to a target tissue/structure such as including a targeted live biofilm, or other biological matter such as blood or interstitial fluid, the target/structure, stained with an appropriate exogenous chromophore, can absorb the intense incandescent energy, thereby causing an increase in temperature in colored or targeted biofilm, changing its nature from a mucinous gel-like fluid to that of a solid coagulum.

In one form of the disclosure, where the optical energy source is a CW (Continuous Wave) diode laser, the optical energy source induces a blackbody “Hot tip” with this secondary incandescent energy transmitted over a distance of free space (propagates) from the source to the tissue/structure, for coagulation and thermolysis of targeted (stained) biofilm, for its destruction/eradication.

Thus, the photons directed from the light source and over free space, which transmission in exemplary embodiments can include use of fiberoptics and a reflector assembly of desired shape and/or a handpiece, the photons are projected forward toward a target region, with a relatively broad beam (compared to a fiber diameter) suitable for application to the sight containing biofilm. In exemplary embodiments of a suitable handpiece or reflector, light can be generally diverted along a propagation or longitudinal axis (e.g., which can be referred to as the “X axis”), with a majority of the light being collimated by the reflector to propagate at least in part along the axis X over free space to the tissue/structure with the biofilm.

Methods and apparatus according to the disclosure can combine the primary laser emissions of conventional near-infrared light sources (e.g., diode) or suitable NIR solid state sources (e.g., Nd:YAG lasers) and the secondary quantum emissions from the incandescent “hot tip” to treat infected tissue and eliminate live biofilms from infected tissue or implanted prosthetic devices. In certain embodiments, other (non-optical) sources, such as electric sources, can be utilized as primary energy sources to carbonize an element (e.g., electrode, conductive element, or fiber) for subsequent secondary (incandescent) energy generation and application to a target site. For example, a current source or free electron laser could be used to cause carbonization or incandescence of an element (e.g., such as a metal filament). The subsequent secondary energy generation could then be directed to a target site for treatment of a (stained) biofilm.

In exemplary embodiments, such an implanted device or infected tissue being treated by the methods of the disclosure can be first treated with a heat sink moiety (or agent or chemical) including such as a dye absorbing electromagnetic energy from an incandescent blackbody radiator.

As mentioned previously, one exemplary biofilm consists of a matrix formed from exopolysaccharide (EPS), water and microbes in percentages of roughly 5% (EPS), 92% (water) and 3% (microbes). The EPS component is an extremely hydrated gel-like (mucinous) bio-polymer that creates a 3-dimensional structure of the biofilm. It is the EPS matrix that protects the microbes within the biofilm from attack by antimicrobial agents (antibiotics) and the immune system. Biofilms and diseased epithelium are highly permeable to Methylene Blue (MB) (and other dyes as described herein). In operation, the intense energy from the incandescent fiber of the therapeutic device of the disclosure is absorbed by MB molecules impregnating the biofilm. That absorbed energy is almost immediately converted to vibrational and rotational energy within the MB molecules. This heat raises the temperature of the MB or anything that is stained with MB.

Accordingly, by means of this method, with the absorption of secondary incandescent energy from the blackbody “Hot Tip”, there is an energy transfer to the live biofilm and diseased epithelium that has been stained with MB. This targeted and controlled heat transfer to the live biofilm produces a semi-solid coagulum from a combination of the mucinous gel-like biofilm and stained diseased epithelium, that can be easily removed with traditional cleaning procedures (for example, root planing and scaling procedures for eliminating biofilms in a periodontal pocket, or other methods of mechanical debridement).

According to a further preferred embodiment of the present disclosure, the primary optical energy source is a Near Infrared Microbial Elimination Laser (NIMEL) system, which can include a dual wavelength solid state near-infrared diode laser system, specifically designed for the purpose of optical bacterial elimination, with minimal heat deposition to the tissue being irradiated. Such NIMELs wavelengths can be utilized to create free radicals such as singlet oxygen in targeted tissue to kill off or mitigate unwanted/undesired microbes (e.g., bacteria, fungus, etc.)

An exemplary embodiment of the Near Infrared Microbial Elimination Laser (NIMEL) system includes an optical radiation generation device, which includes two laser oscillators, one laser oscillator configured to emit optical radiation in a first wavelength range of about 865 nm to about 875 nm, and the other laser oscillator configured to emit radiation in a second wavelength range of about 925 nm to about 935 nm. A delivery assembly preferably including an elongated flexible optical fiber is coupled to the generation device and is adapted for delivery of the dual wavelength radiation from the oscillators to an application assembly. An optical assembly such as a beam expander of a suitable type can be coupled to the light source and/or delivery optics to effectively broaden (e.g., compared to a fiber cross-section) the optical beam propagating from the source/delivery optics. By way of example, such beam expanders can include suitable assemblies of lenses, for example a Keplerian beam expander or a Galilean beam expander.

An optical therapeutic treatment device may include a reflector assembly as disclosed above, for supporting the distal end of the application assembly. In operation, the optical energy introduced by the NIMEL system can propagates, e.g., along an optical fiber and to a reflector/expander, be collimated and then directed to the target area.

Such NIMELs optical radiation can be delivered in one wavelength range (singly), for example, in the first wavelength range of 865 nm to 875 nm, or in the second wavelength range of 925 nm to 935 nm. The radiation in the first wavelength range and the radiation in the second wavelength range also can be combined (or applied successively) or multiplexed, such as by a suitable optical assembly (e.g., prism or “pigtail” fiber assembly) installed in or connected to the optical radiation generation device and delivered to the application site. The NIMEL system, in exemplary embodiments, can utilize a dual wavelength near-infrared solid state diode laser, preferably but not necessarily, in a single housing with a unified control. Preferably, the two wavelengths involve emission in two narrow ranges including 870 nm and 930 nm. In one preferred form, the radiation is substantially at 870 nm and 930 nm, e.g., a desired/sufficient portion of the spectral output is at those wavelengths.

According to an exemplary embodiment of the present disclosure, an infected wound and/or implantable medical structure can be first treated with Targeted Biofilm Thermolysis (in accordance with the current disclosure) at the chromophore stained tissue/structure, which can include a biofilm. Then the thermalized biofilm and diseased tissue on the wound can be cleaned and debrided away. The cleaned wound can then treated with the primary photons from a NIMEL system at a subsequent time. Accordingly, the optical energy generated by the NIMEL system can irradiate or penetrate such a wound/tissue or device/structure, and kill any remaining microbes.

The NIMEL system described is capable of destruction of bacterial cells through the absorption by the bacterial cells of the unique laser energy, selectively in intracellular bacterial chromophores (colors). This can occur without any significant heat deposition. The NIMEL system is able to selectively destroy unwanted biological moieties to a sufficient depth (e.g., bacteria up to four centimeters in soft tissue), while minimizing unwanted hyperthermia (heat and burning) in tissue and surrounding area or medium.

According to a further aspect of the present disclosure, a method for treating a target organism in a region of interest in a patient includes the steps of performing a succession of sub-treatments on the region of interest including a first sub-treatment and a second sub-treatment. The first sub-treatment can include the steps of:

A1. introducing a tissue and biofilm-penetrating material, in one embodiment, MB or another heat sink moiety, to the region of interest, the tissue and biofilm-penetrating material being characterized by optical energy absorption capabilities in a range of treatment wavelength (incandescent light),

A2. providing a first optical fiber having a distal end and a proximal end,

A3. positioning the distal end of the first optical fiber, whereby the distal end is within or adjacent to the region of interest, and

A4. at the proximal and of the optical fiber, introducing optical energy having a predetermined first energy density and a first spectral range into the first optical fiber wherein the introduced optical energy propagates within the first optical fiber from the proximal end to the distal end, and at the distal end, exits the optical fiber, wherein the time integral of the first energy density of the exiting optical energy is sufficient so that the exiting optical energy initially carbonizes the laser fiber, and subsequently causes the carbonized distal tip to incandescently emit optical energy in a second spectral range, wherein the second spectral range is in the range of treatment wavelengths.

The second sub-treatment can include the steps of:

B1. providing a second optical fiber having a distal end and a proximal end,

B2. positioning the distal end whereby the distal end is within or adjacent to the region of interest,

B3. at the proximal end of the second optical fiber, introducing optical energy having a second energy density and a third spectral range into the second optical fiber wherein the introduced optical energy propagates within the second optical fiber from the proximal and to the distal end, and at the distal end, exits the second optical fiber, wherein the third spectral range includes wavelengths within an optical energy absorption range of the target micro-organism(s).

In exemplary embodiments, the time integral of the first energy density of said exiting optical energy is greater than or equal to approximately 600-12,000 Joules/cm² and said first spectral range includes wavelengths in the approximate range of 800 nm to 1100 nm. Additionally, exemplary aspects of the present disclosure can include a reflector of a desired shape, e.g., parabolic, for transmitting incandescent radiation to a desired site, e.g., patient tissue, wound, and/or implantable device. Further, such transmission of radiation can be through free space propagation as opposed to including direct contact of a fiber optic element with the patient tissue or implantable device.

Exemplary embodiments can employ the first sub-treatment alone, which can include the use of non-optical sources such as electrical sources and free electron laser, etc., to cause the secondary (incandescent emission) in the black body tip/element, which as described previously, is not limited to optical fiber structure but can also include other structure(s) and materials.

### BRIEF DESCRIPTION OF DRAWINGS

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIG. 1 is a graph illustrating of a CIE Chromaticity Diagram of an incandescent optical fiber tissue/structure;

FIG. 2A is a diagram illustrating a clean optical fiber tip emitting NIR radiation;

FIG. 2B is a diagram illustrating the secondary optical and thermal energy generated from a carbonized fiber tissue/structure at a delivery site after free space propagation;

FIG. 3. shows a preferred embodiment of the optical therapeutic treatment device according to one aspect of the disclosure;

FIG. 4 shows another preferred embodiment of the optical therapeutic treatment device according to one aspect of the disclosure;

FIG. 5 shows a further preferred embodiment of the optical therapeutic treatment device according to one aspect of the disclosure;

FIG. 6 illustrates a procedure of using the optical therapeutic treatment device shown in FIGS. 3-5 to treat a prosthetic implanted device;

FIG. 7 illustrates a procedure of removing thermolyzed biofilm and tissue coagulum from a prosthetic implant;

FIG. 8 illustrates a diagram of a Near Infrared Microbial Elimination Laser (NIMEL) system according to one preferred embodiment of the present disclosure;

FIG. 9 illustrates a procedure of using the optical therapeutic treatment device shown in FIGS. 3-5 to treat an infected wound;

FIG. 10 illustrates a procedure of removing thermolyzed biofilm and tissue coagulum from a wound after the treatment shown in FIG. 9;

FIG. 11 illustrates a procedure of using the NIMEL system shown in FIG. 8 to treat the wound;

FIG. 12 illustrates a preferred embodiment of a beam expander used with the optical therapeutic treatment device according to one aspect of the disclosure;

FIG. 12A illustrates one preferred embodiment of the beam expander of the optical therapeutic treatment device according to one aspect of the disclosure; and

FIG. 12B illustrates another preferred embodiment of the beam expander of the optical therapeutic treatment device according to one aspect of the disclosure.

While certain embodiments depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

Aspects of the present disclosure capitalize on quantum interactions that can occur when near-infrared (NIR) radiation, such as that generated by suitable diode or solid state lasers, is delivered to a carbonized fiber tip creating a blackbody radiator. The delivery of such incandescent secondary irradiation, which can include free space propagation as opposed to including direct contact of a fiber optic element with patient tissue or implantable device or infected tissues, can produce coagulation and thermolysis of targeted biofilm at or in the delivery site. These quantum and thermodynamic realities can accordingly be exploited by embodiments of the present disclosure to achieve targeted live biofilm thermolysis using near-infrared lasers and the secondary quantum emissions.

The present disclosure discloses, inter alia, novel techniques and devices for exploiting the (near instantaneous) transformation of radiation into an incandescent blackbody radiator, e.g., as NIR radiation produced by diode and/or solid state NIR laser media, and delivery of such radiation to structure that acts a as a black body radiator. For example, NIR radiation can be supplied to an end of an optical fiber, which can thereby become a carbonized fiber tip. The resulting incandescent blackbody radiator can produce secondary energy emission that can be utilized to deleteriously effect the targeted biofilm(s) at the site; such secondary energy emission may further be capable of cutting and vaporizing adjacent tissues. Accordingly, such transmission techniques and incandescent blackbody radiators have been found by the inventor to have quantum and thermodynamic properties useful for the treatment of diseased tissues and implantable devices that include in particular the reduction of live biofilms.

When photons of a desired incandescent spectrum are transmitted, e.g., after being collimated and then sent through free space propagation, to a target tissue including a live biofilm, or other biological matter such as blood or interstitial fluid, the stained/targeted biofilm and surrounding tissue/structure can absorb the secondary incandescent radiation thereby causing an increase in temperature and thermolysis of the biofilm.

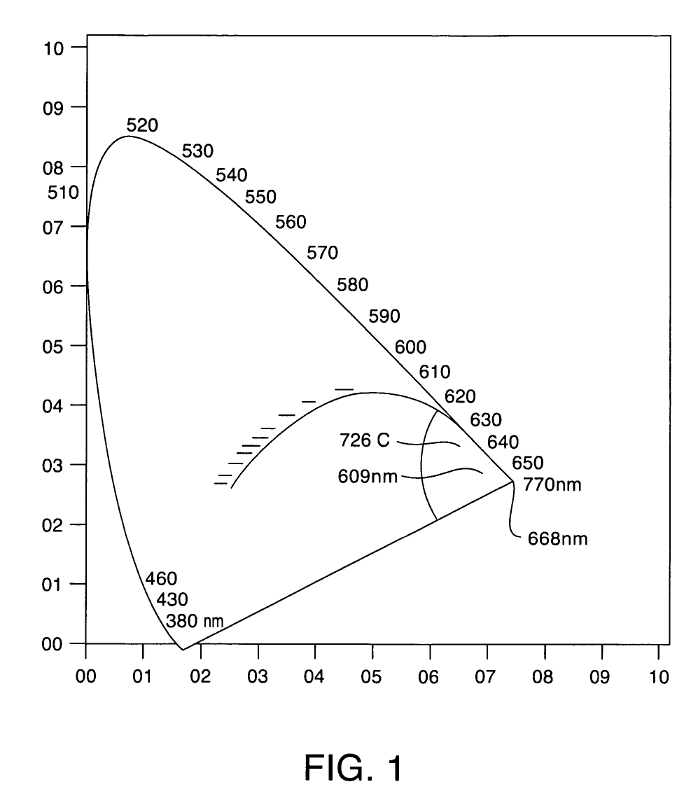

As the hot tip begins to glow with heat, it emits first red, and then orange visible light. This can be evidenced by a CIE Chromaticity Map overlaid with a black body locus as shown in FIG. 1, as the incandescent tip reaches (900° C. to 1200° C.). As the primary energy of the laser is converted from one form into another, a portion of the energy in the transfer becomes energy in a diffuse form (in this example heat) rather than the straightforward primary photons from the laser.

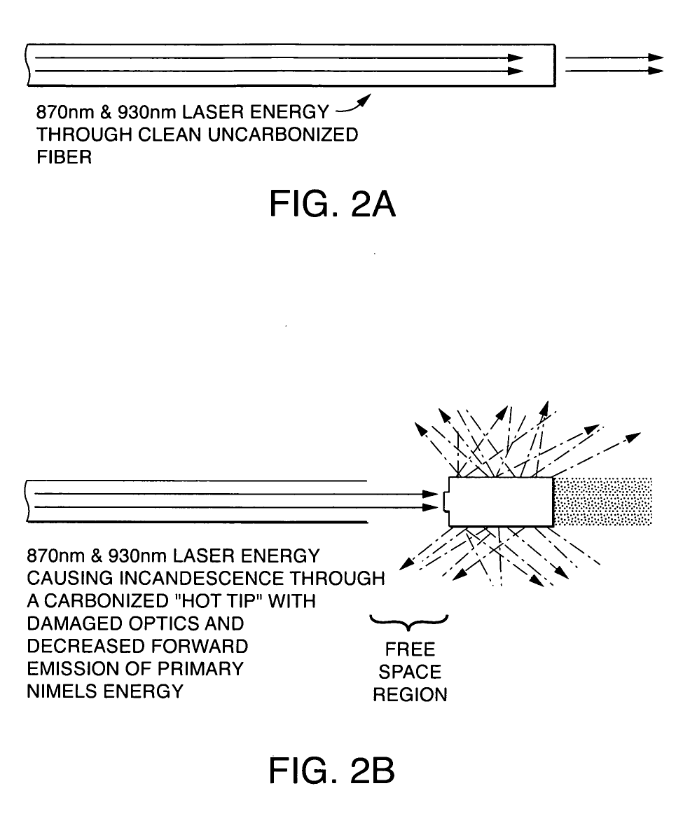

FIG. 2A shows an example of free space propagation of NIMELs wavelengths from an end of a fiber optic waveguide (optical fiber) according to an embodiment of the present disclosure. As shown in FIG. 2A, the light emitted from a fiber distal end (with the NIR source to the left, not shown) has a degree of collimation when emitted towards a target tissue/structure for subsequent Biofilm Thermolysis, that is not available with the incandescent fiber alone. FIG. 2B depicts a view of NIMELs radiation sent from the end of fiber and over a distance of free space to a target tissue/structure, where as shown in FIG. 2B, the secondary emission at the target tissue/structure can be diffuse.

The photobiology of the “incandescent hot tip” phenomenon with near-infrared diode lasers has been explained above. As shown in FIG. 2B, the incandescent energy generated at the hot tip can be diffused, e.g., with less than optimal optical qualities for delivery to biofilm impregnated areas. Exemplary embodiments of the present disclosure present a solution to focus the secondary incandescent emission in a straight and direct and relatively broad beam vector for delivery to a site so as to effect live biofilm thermolysis on infected joints and/or other prostheses, and/or infected tissue.

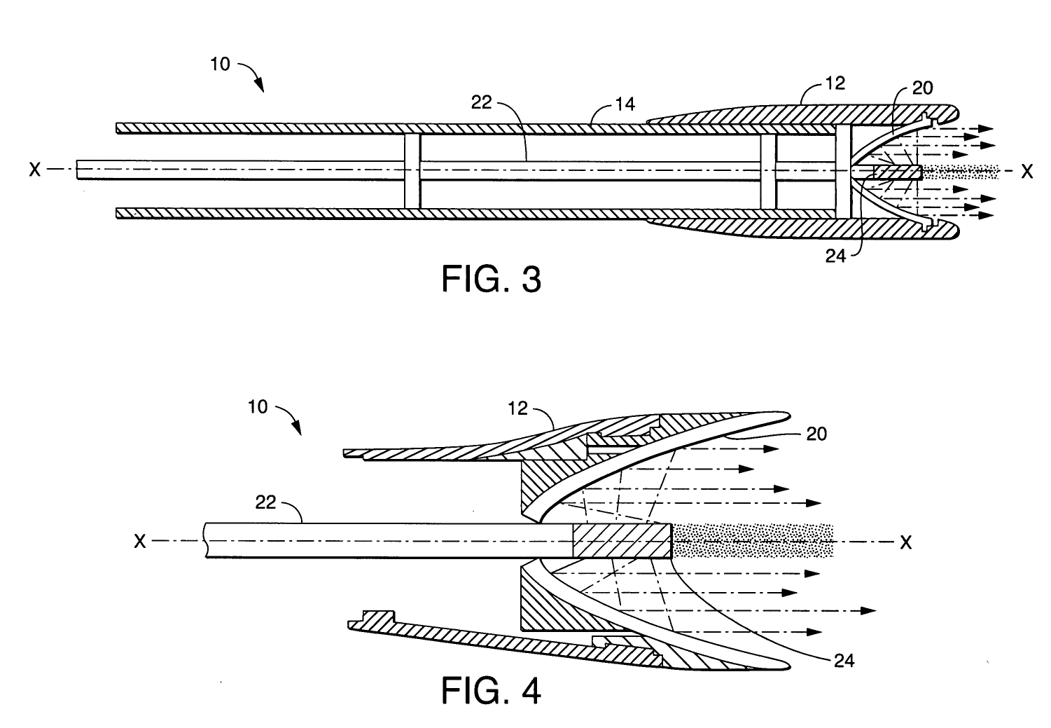

According to one preferred embodiment as shown in FIG. 3, the optical therapeutic treatment device 10 includes a housing 12 extending along a central axis X, an elongated fiber guide 14 at least partially received in the housing 12 and adapted to receive an optical fiber having a proximal end and a distal end 24, a reflector assembly 20 within the housing and extending along the central axis X. In one form of the disclosure the fiber is “removable” and “replaceable” with respect to the housing. In other forms, the distal end of the fiber is fixedly attached to the housing. The distal end is carbonized by the radiation from the source.

As shown in FIG. 3, the fiber guide 14 can be adapted to position a distal end 24 of an optical fiber 22 received therein, to be aligned with the central is X and within the reflector assembly 20. The reflector assembly 20 preferably has a parabolic cross-section taken along the central axis X. The reflector assembly 20 is adapted to reflect optical energy propagating radially with respect to the central is X, so that the reflected optical energy propagates at least in part along a propagation axis parallel to the central axis X, effecting a relatively broad (compared to fiber diameter) “beam”.

According to a further embodiment, the optical therapeutic device 10 can further include an optical energy source and an associated coupling assembly for introducing optical energy generated by the source to a proximal end of the optical fiber 22. The introduced optical energy propagates within the optical fiber 22 to the distal end 24 thereof and exits the optical fiber 22 at the distal end 24. The distal end 24 is preferably removably coupled to the fiber guide 14, and thereby the assembly including the housing 12, the fiber guide 14, and the reflector assembly 20 can be removed from the fiber 22 and is disposable. The distal end 24 also can be fixedly coupled to the fiber guide 14.

The optical therapeutic device 10 may include more than one optical fiber (e.g., a bundle of fibers) each extending along an axis parallel to the central axis X and having its proximal end coupled to the optical energy source and distal end coupled to the fiber guide 14 and positioned within the reflector assembly 20. The coupling assembly can be adapted for coupling the generated optical energy to the proximal end of one of the optical fibers when the distal end of said one optical fiber is received within the reflector assembly or be adapted for coupling the energy to the proximal end of all the optical fibers.

As shown in FIG. 3, the reflector 20 defines a central bore for receiving the distal end 24 of the optical fiber 22, which can be shaped as desired to emit radiation supplied from an NIR source (e.g., to the left drawing, not shown). The distal end 24 can be positioned such that the parabolic reflector 22 reflects the NIR energy and projects it forward toward a target region for biofilm thermolysis at the region and subsequent mitigation of unwanted biological moieties such as coagulated biofilm.

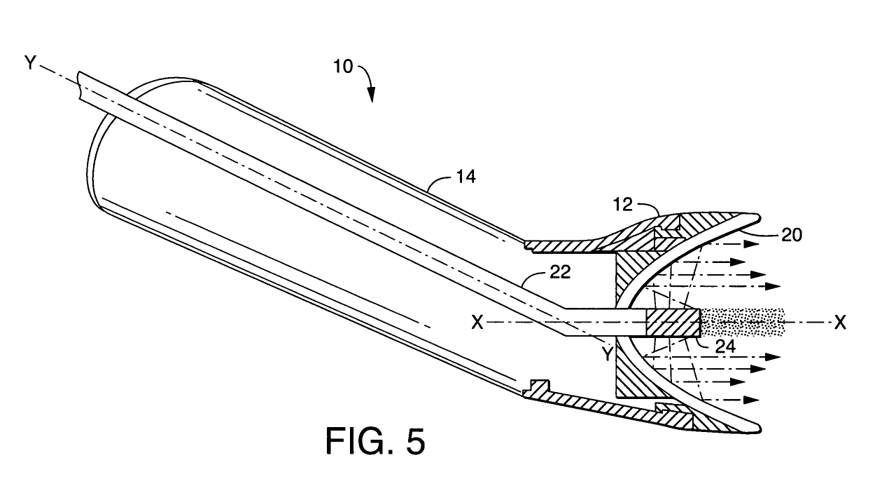

FIGS. 4 and 5 show two more different exemplary configurations of the optical therapeutic treatment device, which are similar to the embodiment shown in FIG. 3, such that similar elements use same reference numerals. In FIG. 4, the optical therapeutic treatment device 10 includes a housing 12 extending along a central axis X and a reflector assembly 20 within the housing and extending along the central axis X. The reflector 20 defines a central bore through which an optical fiber 22 is inserted. The reflector assembly 20 preferably has a parabolic cross-section taken along the central axis X. Other reflector geometries (e.g., hyperbolic, elliptical, etc.) may be used in other embodiments. The distal end 24 of the optical fiber is positioned within the reflector 20 such that the parabolic reflector 20 reflects the produced energy and projects it forward toward a target region.

FIG. 5 shows a further preferred embodiment, in which the optical therapeutic treatment device 10 includes a housing 12 extending along a central axis X between a proximal end and a distal end, an elongated fiber guide 14 extending along a central axis Y between a proximal end and a distal end, where the distal end of the fiber guide 14 is coupled to the proximal end of the housing 12, and a reflector assembly 20 within the housing and extending along the central axis X. The axis Y and the axis X form an angle, which can be adjustable or be different in different embodiments as required by the practitioner for different procedures. The fiber guide 14 receives and guides an optical fiber 22 through the proximal end to the distal end of the fiber guide 14. The reflector 20 defines a central bore through which a distal end 24 of the optical fiber 22, which has a carbonized distal tissue/structure, passes through. The reflector assembly 20 has a parabolic cross-section taken along the central axis X, such that the parabolic reflector 20 reflects energy applied to the target site, e.g., incandescent energy generated by the incandescent distal end 24 (for embodiments utilizing a carbonizing fiber tip) positioned within the reflector 20 and projects it forward toward a target region.

According to a further embodiment, the optical therapeutic treatment device can include only the reflector assembly, which is adapted to be coupled to the optical fiber with the distal end positioned within the reflector assembly. The reflector assembly can reflect the incandescent energy generated by the carbonized distal end and project it forward toward a target region.

The methods and apparatus according to the disclosure can be utilized to combine the primary emissions of NIR light sources (e.g., one or more diode lasers) and the secondary quantum emissions from the optical energy source used according to the disclosure to treat infected tissue and live biofilms formed on implanted prosthetic devices.

A large number of laser sources in the infrared spectrum have been used to kill pathogenic bacteria. For the last few years near infrared solid state diode and Nd:YAG lasers have been used in the field of dentistry for tissue cutting, cautery, and bacterial thermolysis. The four most widely used near infrared wavelengths are 810 nm, 830 nm, 980 nm and 1064 nm. These near infrared lasers have very low absorption curve in water, and have a very deep tissue penetration values as detailed infra.

An aspect of the disclosure provides novel apparatus and methods for the treatment of infected implanted devices. Such an implant being treated by the methods of the disclosure can be treated with targeting a biofilm with a heat sink moiety, e.g., a dye absorbing at an incandescent tip's spectral range. The term “predetermined spectral range” can include the range of an incandescent blackbody radiator and the range of from about 800 nm to about 1100 nm for the primary energy device. A “heat sink” moiety can be any entity capable of receiving, absorbing or otherwise diverting heat from the tissue being irradiated with the optical energy source. Heat sink moieties according to the disclosure include compounds known to act as chromophore dyes (i.e., molecules that preferentially absorb optical energy).

Representative examples of chromophore dyes include Toludine Blue (with absorption spectra between 600 nm to 700 nm), Methylene Blue (MB, with absorption peaks at 609 nm (orange) and 668 nm (red)), Congo Red (with strong absorption band at 340 nanometers in the near-ultraviolet region and another at 500 nanometers near the blue-green transition region), and Malachite Green (with a strong absorption band centered at 600 nanometers near the yellow-red transition region, and any other tissue safe biological dye). One of skill will appreciate that chromophore dyes may be administered in a composition form including any known pharmacologically acceptable vehicle with any of the well known pharmaceutically acceptable carriers, including diluents and excipients (see Remington's Pharmaceutical Sciences, 18‘x’ Ed., Gennaro, Mack Publishing Co., Easton, Pa. 1990 and Remington: The Science and Practice of Pharmacy, Lippincott, Williams & Wilkins, 1995). Formulations of compositions according to the disclosure may contain more than one type of chromophore dye according to the disclosure), as well any other pharmacologically active ingredient useful for the treatment of the symptom/condition being treated. Hence, in some instances, the practitioner may opt to co-administer other active or inactive components including, but not limited to, antibiotics, anesthetics, and flavorants.

Examples of useful antibiotic or antimicrobial agents include, but are not limited to, chlorhexidine gluconate, triclosan, cetyl pyridinium chloride, cetyl pyridinium bromide, benzalkonium chloride, tetracycline, methyl benzoate, and propyl benzoate. Examples of useful anesthetic agents include, but are not limited to, benzocaine, lidocaine, tetracaine, butacaine, dyclonine, pramoxine, dibucaine, cocaine, and hydrochlorides of the foregoing. Suitable flavorants include, but are not limited to, at least one of peppermint oil, sodium saccharine, aspartame, oil of wintergreen, oil of spearmint, strawberry favoring, and grape flavoring.

In the following examples in this disclosure, MB (for example, 1% solution) is used as a heat sink moiety. MB has been used previously in medicine as an oxidation reduction indicator, an antidote to cyanide, and as a mild antiseptic. In biophotonics, MB has been used primarily as a photo-sensitizer for individual bacteria with (soft) low level visible red lasers (laser output power of 100 mW or less).

One type of biofilms consists of a matrix formed from exopolysaccharide (EPS), water and microbes in percentages of roughly 5% (EPS), 92% (water) and 3% (microbes). The EPS component is an extremely hydrated gel-like (mucinous) bio-polymer that creates a 3-dimensional structure for the biofilm. It is the EPS matrix that protects the microbes within the biofilm from attack by antimicrobial agents (antibiotics) and the immune system. Biofilms and diseased epithelium are highly permeable to MB. In operation, the biofilm (where the bacteria live) with a heat sink (MB) for thermolysis are targeted by the incandescent radiant energy.

Given the above targeting mechanism of the biofilm, it then follows that the intense heat energy (e.g., from the incandescent fiber tip or tissue/structure) can be absorbed by such heat sink moieties (or chromophores) such as MB molecules impregnating the biofilm, and are then almost immediately converted to vibrational and rotational energy within the MB molecules, which is the molecular basis for heat. This heat raises the temperature of the MB or anything that is stained with MB. Accordingly, by means of this method, with the absorption of the secondary incandescent energy from the incandescent fiber, there is an energy transfer to the live biofilm and diseased infected tissues that have been stained with MB. This targeted and controlled heat transfer to the live biofilm produces a semi-solid coagulum from the biofilm and stained diseased epithelium, that can be easily removed with traditional cleaning procedures (for example, root planing and scaling procedures in a periodontal treatment).

With this method, a practitioner can easily accomplish a live biofilm phase change through coagulation and thermolysis of the gel-like matrix of the biofilm. This will lead to safer and more successful procedures for patients with infected prosthetic implants or infected tissue, and preserve more healthy collagen, bone, and mucosa in the area from irreversible thermal damage during the procedure, while at the same time, facilitating the removal of the biofilm on the prosthetic implants or infected tissue. Once the biofilm is removed, the previously infected area has the immediate potential to heal.

In the exemplary embodiment, MB is used as a heat sink moiety, however, a person skilled in the art should understand that other heat sink moieties also can be used. Preferably, a heat sink moiety is: (i) essentially non-toxic or minimally toxic to tissues; (ii) able to penetrate live biofilm; and (iii) selectively absorbed by the live biofilm to target the same without damaging the tissues of the patient.

Treatment Time

To allow for safe and efficient radiant heat to be applied to the target area and prevent unwanted tissue from being damaged, calculating and controlling the dosage of the radiation of high importance. The following equations present calculations of dosimetry for “primary photon” such as released from the optical reflector or optical fiber, etc.

In the equations, the output power of a laser device, refers to the number of photons emitted from the laser at a given wavelength and is measured in Watts.

The power density of a laser beam measures the potential thermal effect of laser photons at a treatment irradiation site/area of tissue. The power density (W/cm²) is a function of the output power and the beam area (the area of the cross-section perpendicular to the propagation direction of the beam) as shown in the following equation:

$$Power Density ( W / {cm}^{2} ) = \frac{Laser Output Power ( W )}{Beam Area ( {cm}^{2} )} \qquad 1 )$$

The total energy delivered on a target area by a laser system operating at a particular output power over a certain period of time is measured in Joules and is obtained with the following equation:
Total Energy(Joules)=Laser Output Power(Watts)×Time(Sec) 2)

It is essential to know the distribution and allocation of the total energy (Joules) delivered onto the target region, in order to correctly measure tissue site dosage for maximal beneficial tissue response. Total energy distribution can be measured as energy density in (Joules/cm²). The energy density is a function of power density and time, and is measured in (Joules/cm²) and is calculated as follows:
Energy Density(Joules/cm²)=Power Density(W/cm²)×Time(sec) 3)

Usually, for the “primary photon” released from the optical fiber (without a carbonized incandescent distal tip and incandescent energy), to calculate the treatment time to deliver a dose of laser energy to a given volume of tissue, for a given tissue response, a practitioner needs to know either the energy density (J/cm²) or total energy (J) for said given tissue response, as well as the output power (W), and beam area (cm). Then, the treatment time can be calculated with the following equation:

$$Treatment Time ( seconds ) = \frac{Energy Density ( Joules \text{/} {cm}^{2} )}{Power Density ( W \text{/} {cm}^{2} )} \qquad 4 )$$

For the optical therapeutic treatment device of the present invention, with the carbonized incandescent tip at the distal end of the diode laser fiber, it is very difficult to precisely calculate the value of the “beam area”, and hence, there is no practical “power density” and/or “energy density” equation to calculate. Therefore, the treatment time can be estimated or rely on the following equation (4a):

$$Treatment Time ( seconds ) = \frac{Total Energy ( Joules )}{Output Power ( Watts )} \qquad 4 a )$$

For an optical energy source, the output power is known. For a particular desired total energy that a practitioner plans to be applied onto a target area, the practitioner can calculate the needed treatment time with the novel above (4a) equation. This incandescent energy dose can be used to target a stained biofilm in or on an infected area based on the parameter of time, to achieve live biofilm coagulation. These parameters can also be manipulated with CW diode lasers by additional clinical modifications that simply involve an increase or decrease of the total energy value for a given biofilm targeting procedure to make the incandescent tip emissions greater or smaller. For example, altering the value of total energy to perform safe procedures with the incandescence phenomenon can be simply accomplished by increasing or decreasing the laser output power. The dosimetry calculation is dependent on the wound or infected area to be irradiated. In operation, a practitioner can simply manipulate both laser output power and/or treatment time in a treatment to ensure maximum safety and successful treatment with CW diode lasers.

Biofilm Eradication Technology for Treatment of Infected Prosthetic Joints

Infections that occur with artificial joint replacements are categorized as either acute or chronic. Acute infections develop with the first three weeks of surgery. Bacteria can enter the surgery region during implantation or through breaches in the skin and form a biofilm on the implanted prosthetic joint. If wound healing problems and local tissue necrosis are present, acute infections are more likely to happen. Chronic infections will surface months or years after surgery. Chronic infections can result from the transient presence of bacteria in the blood stream (e.g., from periodontal disease) that will then cause biofilm formation on the implanted prosthetic device.

Transient bacteria can also be associated with distant infections involving urinary tract, lungs or skin. To prevent these problems, individuals with total joint replacements are often encouraged to take antibiotics prior to dental surgery, colonoscopy and other procedures where organisms are frequently released. In many cases no identifiable cause is present; however, immune compromise, rheumatoid arthritis, diabetes, and obesity are all considered to be risk factors.

Infections that are diagnosed within several weeks of surgery are possibly controlled with joint irrigation and suppressive antibiotics. It is difficult for the body's immune system to remove well established infections from the surface of artificial materials because of the mature biofilms coating the prostheses. This fact makes the prosthetic joint infections difficult to cure. Current treatment options show marginal efficacy and are individualized to the specific patient.

The most frequently utilized approach is a two-stage implant exchange (i.e. new joint surgery). In the initial stage, the implant and the cement are removed along with all infected bones, soft tissue, and joint lining (synovectomy). The region is irrigated with a large volume of solution after which an antibiotic impregnated spacer is placed. The patient typically receives a 6-week course of antibiotics and the joint is aspirated prior to any further surgery to evaluate for recurrent infection. If the infection has been cleared, then a new joint prosthesis can be implanted.

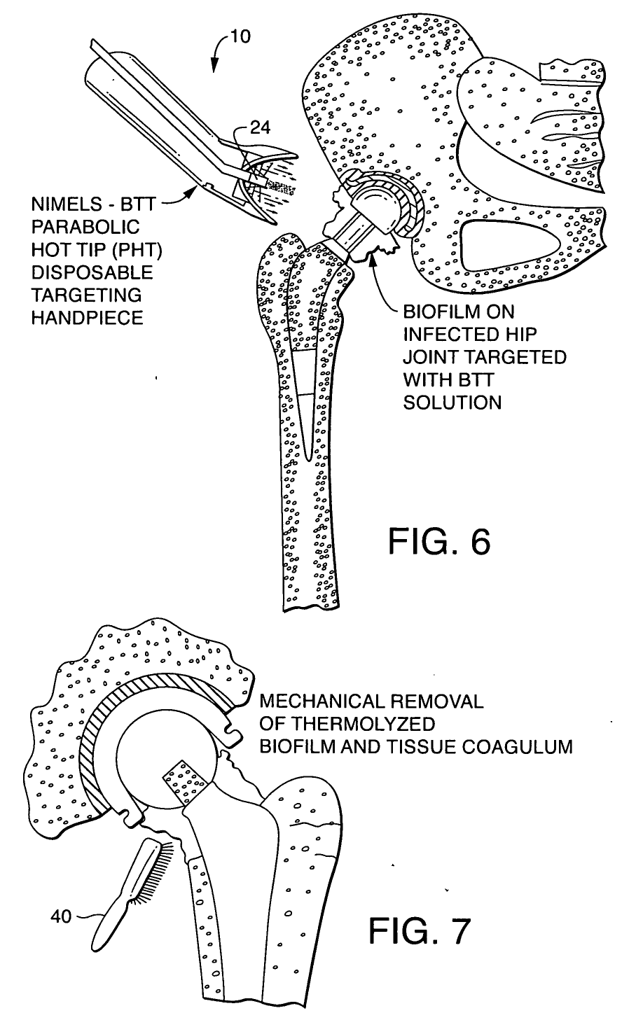

According to the present disclosure, in treatment, once the infected prosthesis is surgically opened and revealed, the infected area would be saturated with a MB solution or spray (or other targeting chromophore) to target the biofilm and diseased tissue. As shown in FIG. 6, the optical therapeutic treatment device 10 with the carbonized fiber tip 24 disposed therein is then placed close to the target region. The incandescent energy is then applied to the target region.

In operation, once the biofilm on the prosthetic implants is successfully thermolyzed, mechanical debridement of the newly formed semi-solid coagulum can be accomplished with a cleaning procedure. In one preferred form, a biofilm and tissue debridement brush 40 as shown in FIG. 7 is employed in the surgical procedure to precisely remove thermolyzed biofilm and infected soft tissue in the form of a semi-solid coagulum from the infected area. In this procedure, the biofilm and tissue coagulum is abraded under irrigation using the soft and pliable surface of the debridement brush 40. In another preferred form This biofilm debridement brush can be coupled to any ultrasonic and/or irrigation system to aid in the removal of thermolyzed biofilm and diseased tissue. This can be accomplished without causing any further mechanical damage to the prosthesis or surrounding healthy tissue. Once the thermolyzed biofilm and tissue coagulum is removed and well irrigated, the area can be closed, with appropriate antimicrobials and drains applied. The infected prosthesis and the area should then heal normally.

Biofilm Eradication Technology for Treatment of Infected Wound

Traditionally solid state diode lasers in the visible and near infrared spectrum (600 nm to 1100 nm) have been used for a variety of purposes in medicine, dentistry, and veterinary clinic because of their preferential absorption curve for melanin and hemoglobin in biological systems. They rarely, if at all, have been used for sterilization outside of biological systems. Near infrared (NIR) energy (such as diode laser energy) can typically penetrate biological tissue to about four centimeters. Thus, with optical radiation from near infrared diode lasers, heat deposition is much deeper in biological tissue than it is with the mid-infrared wavelengths. However, to prevent unwanted thermal injury to healthy tissue in a biological site being irradiated (e.g. site of infection), the radiance (joules/cm²) and/or the exposure time of primary photons from near infrared lasers must be kept to a minimum value.

For the destruction of bacterial cells with visible and near infrared diode lasers, the prior art conventionally requires the presence of an exogenous chromophore in a site being irradiated and provides a very narrow therapeutic window and opportunity for treatment, as a five- to ten-second duration of temperature above 80° C. will irreversible harm healthy cells. Photothermolysis (heat induced death) of bacteria with near infrared laser energy, in the prior art, requires a significant temperature increase that may endanger healthy cells. It is generally desired to destroy bacteria thermally, without causing irreversible thermal damage to healthy cells.

The Near Infrared Microbial Elimination Laser (NIMEL) system, as shown and described herein, can include a dual wavelength solid state near-infrared diode laser system, specifically designed for the purpose of optical bacterial elimination, with minimal heat dissipation in the tissue being eradiated. Such NIMEL applications and techniques can produce photo-absorption in biological moieties producing production of toxic free radicals or reactive oxygen species.

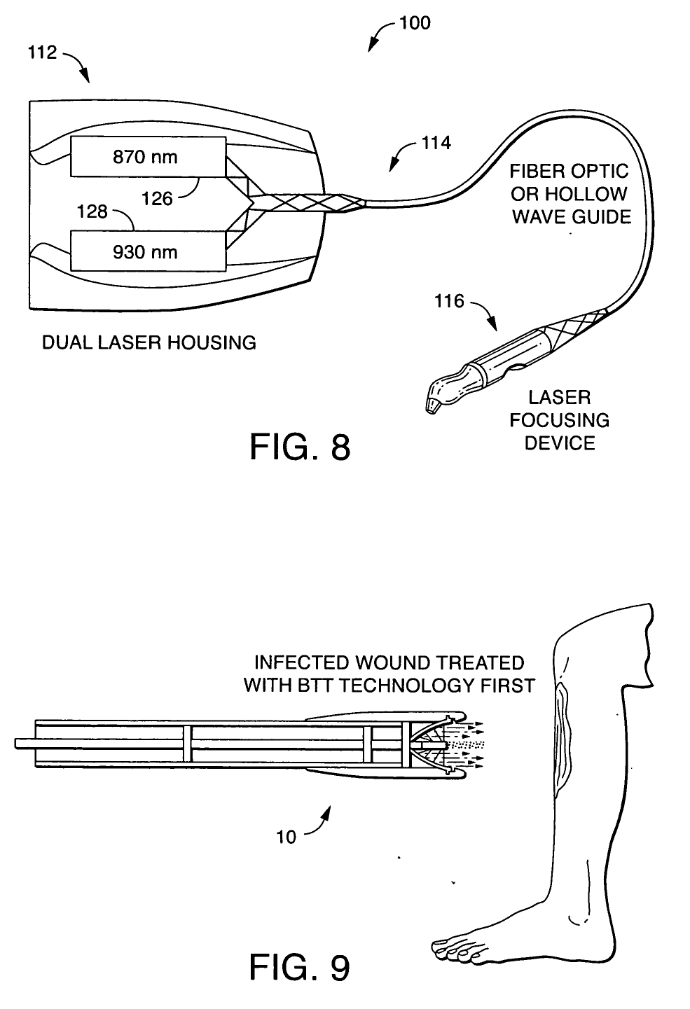

One exemplary embodiment of the Near Infrared Microbial Elimination Laser (NIMEL) system is shown in FIG. 8. The NIMEL system 100 includes an optical radiation generation device 112, a delivery assembly 114, and an application assembly (or region) 116. The optical radiation generation device 112 includes laser oscillators 126 and 128, one laser oscillator 126 configured to emit optical radiation in a first wavelength range of about 865 m to about 875 m, and the other laser oscillator 128 configured to emit radiation in a second wavelength range of about 925 nm to about 935 nm. The delivery assembly 114 preferably includes an elongated flexible optical fiber adapted for delivery of the dual wavelength radiation from the oscillators 126 and 128 to the application assembly 116.

According to one aspect of the present disclosure, an optical therapeutic treatment device 10 as shown in FIGS. 3-5 (not shown in FIG. 8) is placed at a distal end of the application assembly 116; however, in this configuration, an optical fiber 22A (without a carbonized tissue/structure) is within the housing 12. Preferably, an optical beam expander assembly 200 is coupled to the distal tissue/structure of the fiber. Preferably again, the optical fiber 22A and beam expander assembly 200 are removably placed within the housing 12 so that the same housing might be sequentially used with a fiber 22 that forms a carbonized hot tip as noted above for BTT process and then used with a fiber 22A with beam expander 200 for a NIMEL process.

In operation for the NIMEL process as shown in FIG. 8, the optical energy propagates along the optical fiber 22A to the distal tissue/structure, and through the beam expander 200, and onto the target area.

The optical radiation can be delivered in one wavelength range only, for example, in the first wavelength range of 865 nm to 875 nm, or in the second wavelength range of 925 nm to 935 nm. The radiation in the first wavelength range and the radiation in the second wavelength range also can be multiplexed by a multiplex system installed in the optical radiation generation device 112 and delivered to the application site in a multiplexed form. The NIMEL system, in one form, may utilize a dual wavelength near-infrared solid state diode laser, preferably but not necessarily, in a single housing with a unified control. The two wavelengths involve emission in two narrow ranges approximating 870 nm and 930 nm. In one preferred form, the radiation is substantially at 870 nm and 930 nm. The dosimetry for the NIMEL process can be selected such that a high enough power density is applied to the target area to effect killing of bacteria, but not so high that eukaryotic cells are killed.

The NIMEL system is capable of destruction of bacterial cells through the absorption by the bacterial cells of the unique laser energy, selectively in intracellular bacterial chromophores (colors). This will occur without the significant deleterious heat deposition to the tissues being irradiated. The NIMEL system is accordingly able to selectively destroy bacteria up to four centimeters in soft tissue, while minimizing the unwanted hyperthermia (heat and burning) of the lased tissue and surrounding area or medium, thereby greatly improving the infection fighting potential of the laser.

With less heat deposition to the system being irradiated, there is a broad application range for NIMEL technology, which include fields of human and veterinary medicine and dentistry, laboratory biology and microbiology, food service, and any other area needing bacterial control without the unwanted side effects of ionizing radiation, ultraviolet light, and excessive heat deposition.

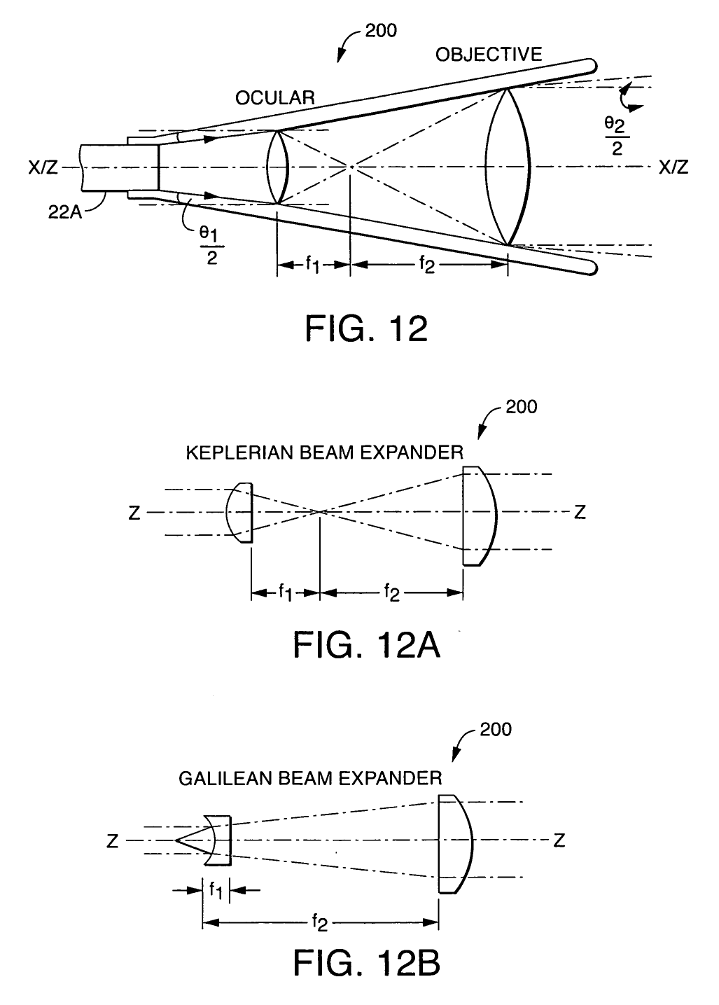

In one preferred embodiment as shown in FIGS. 12, 12A and 12B, the optical therapeutic treatment device includes a beam expander 200, which is designed to take a small-diameter collimated input beam and produce a larger diameter collimated output beam, thus reducing the divergence of the beam. The beam expander 200 extends along a longitudinal is Z. The beam expander 200 is connected to the distal tissue/structure of the fiber 22A such that the longitudinal axis Z of the beam expander 200 is coaxial with the central is X of the reflector assembly 20. The optical energy at the distal end 24 of the fiber 22A forms a collimated energy beam which propagates along the is X and enters the beam expander 200 at the input end of the beam expander 200. The beam expander 200 increases the diameter of the cross-section perpendicular to the propagation direction of the energy beam at the output end of the beam expander 200.

The beam expander 200 can be any optical system designed to increase the diameter of a laser beam. There are two basic types of beam expander which can be used in the optical therapeutic treatment device of the present disclosure. One type is Keplerian beam expander, as shown in FIG. 12A, which includes a positive input lens and a positive objective lens separated by the sum of their focal lengths. The other type is Galilean beam expander, as shown in FIG. 12B, which includes a negative input lens and a positive objective lens separated by the difference of their focal lengths. Other beam expanders also can be used with the present disclosure.

### EXAMPLE 1

### Treatment of an Infected Wound

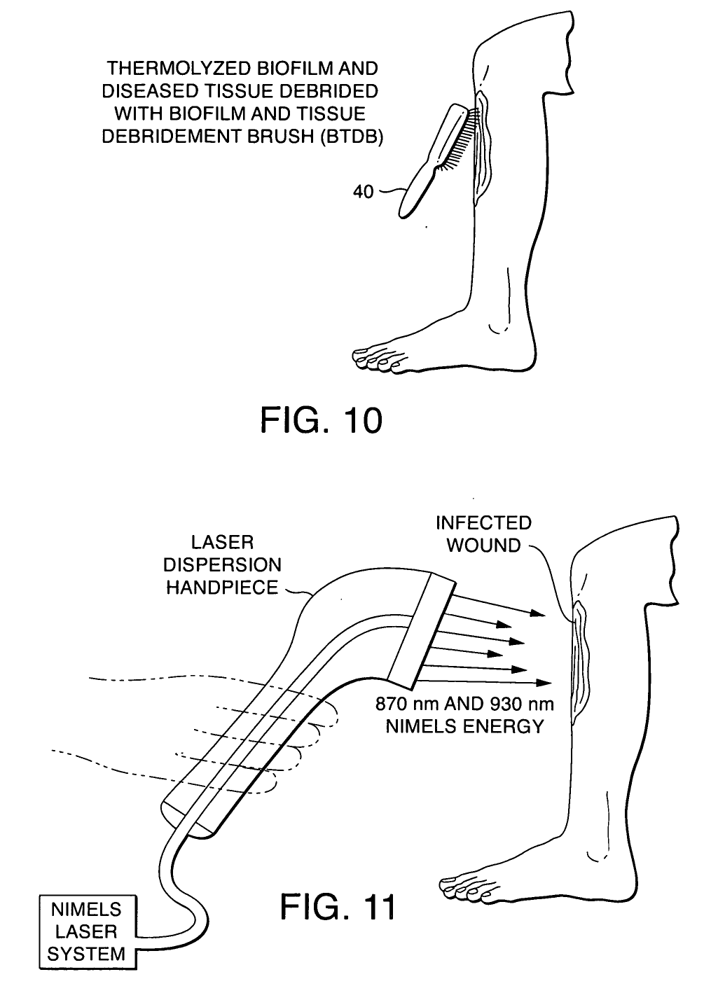

On day one of the treatment, the biofilm in the wound or foot-ulcer is dyed with Methylene Blue solution or equivalent, and the output power of a NIMEL system is increased enough to generate an “incandescent tip” in an optical therapeutic treatment device 10 for targeting live biofilms, as shown in FIG. 9. The incandescent energy generated at the incandescent tip is then applied to the target area. The area is then debrided and irrigated with the tissue debridement brush 40 as shown in FIG. 10.

On day two through seven of treatment, the optical power (i.e. the primary NIMEL photons with minimal heat deposition) of the NIMEL system is employed (through a different handpiece) to penetrate healing wound and kill any remaining bacteria, as shown in FIG. 11, thereby allowing tissue healing, with the dosimetry calculated with the algorithm disclosed above.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

While the disclosure is exemplified in human patients, the methods and apparatus of the present disclosure are intended for use with any mammal that may experience the benefits of the method and apparatus of the disclosure. Foremost among such mammals are humans, although the disclosure is not intended to be so limited, and is also applicable to veterinary uses. Thus, in accordance with the disclosure, “mammals,” or “mammal in need,” or “patient” include humans as well as non-human mammals, particularly domesticated animals including, without limitation, cats, dogs, and horses.

While the claimed disclosure has been described in detail and with reference to specific embodiments thereof, it will be apparent to one of ordinary skill in the art that various changes and modifications can be made to the claimed disclosure without departing from the spirit and scope thereof. Thus, for example those skilled in the art will recognize, or be able to ascertain, using no more than routine experimentation, numerous equivalents to the specific substances and procedures described herein. Such equivalents are considered to be within the scope of this disclosure, and are covered by the following claims.

What is claimed is:

1. An optical therapeutic treatment device comprising:
A. a housing extending along a central axis;
B. a fiber guide coupled to said housing adapted to receive an optical fiber having a proximal end and a distal end; and
C. a reflector assembly within said housing and extending along said central axis,
wherein said fiber guide is adapted to position the distal end of an optical fiber received therein, to be aligned with said central is and within said reflector assembly, and
wherein said reflector assembly is adapted to reflect optical energy propagating radially with respect to said central axis, so that said reflected optical energy propagates at least in part along a propagation axis parallel to said central axis.

2. An optical therapeutic device according to claim 1 further including an optical fiber having a proximal end and a distal end, with said distal end received in said fiber guide.

3. An optical therapeutic device according to claim 2, wherein said distal end of said fiber includes a carbonized distal hot tip.

4. An optical therapeutic device according to claim 2 further comprising an optical energy source and an associated coupling assembly for introducing optical energy generated by said source to said proximal end of said fiber, whereby said introduced optical energy propagates within said optical fiber to said distal end thereof and exits said optical fiber at said distal end.

5. An optical therapeutic device according to claim 4, wherein said optical energy is coherent.

6. An optical therapeutic device according to claim 4, wherein said optical energy is non-coherent.

7. An optical therapeutic device according to claim 4, wherein said optical energy source is adapted to generate optical energy in a near infrared spectrum range from about 800 m to about 1100 m.

8. An optical therapeutic device according to claim 4, wherein said optical energy source includes two laser oscillators, one laser oscillator configured to emit optical radiation in a first wavelength range of about 865 nm to about 875 nm, and the other laser oscillator configured to emit radiation in a second wavelength range of about 925 nm to about 935 nm.

9. An optical therapeutic device according to claim 2 wherein said distal end is fixedly coupled to said fiber guide.

10. An optical therapeutic device according to claim 2 wherein said distal end is removably coupled to said fiber guide.

11. A kit comprising the optical therapeutic device according to claim 1, and two or more optical fibers, each having a proximal end and a distal end, and each being adapted to be received within said fiber guide and each distal end being adapted to be positioned within said reflector assembly.

12. A kit according to claim 11 further comprising an optical energy source for generating optical energy in a predetermined spectrum, and a coupling assembly for coupling said generated optical energy to the proximal end of one of said optical fibers when said distal end of said one optical fiber is received within said fiber guide.

13. An optical therapeutic device according to claim 1, wherein said reflector assembly has an inner surface having a substantially parabolic cross-section taken along said central axis.

14. An optical therapeutic treatment device according to claim 1, wherein the reflector assembly is configured and arranged for free space forward transmission of secondary blackbody (Incandescent) radiation to a target site.

15. An optical therapeutic treatment device according to claim 14, further comprising a lens configured and arranged to receive light from an inner surface of the reflector assembly for forward free space transmission of secondary blackbody Incandescent radiation to a target site.

16. An optical therapeutic device comprising:
A. an optical fiber extending between a proximal end and a distal end, the proximal end being adapted to receive optical energy incident thereon, the optical fiber being adapted to transmit the received optical energy to the distal end; and
B. a reflector assembly extending along a central axis and coupled to the distal end of the optical fiber, with said distal end positioned within said reflector assembly,
wherein said reflector assembly is adapted to reflect the optical energy emitted from said distal end and propagating radially with respect to said central axis, so that said reflected optical energy propagates at least in part along a propagation axis parallel to said central axis.

17. An optical therapeutic device according to claim 16, wherein said distal end of said optical fiber includes a tip capable of being carbonized or a carbonized hot tip.

18. An optical therapeutic device according to claim 16 further comprising an optical energy source and an associated coupling assembly for introducing optical energy generated by said source to said proximal end of said fiber, whereby said introduced optical energy propagates within said optical fiber to said distal end thereof and exits said optical fiber at said distal end.

19. An optical therapeutic device according to claim 18, wherein said optical energy is coherent.

20. An optical therapeutic device according to claim 18, wherein said optical energy is non-coherent.

21. An optical therapeutic device according to claim 18, wherein said optical energy source is adapted to generate optical energy in a near infrared spectrum range from about 800 nm to about 1100 m.

22. An optical therapeutic device according to claim 18, wherein said optical energy source includes two laser oscillators, one laser oscillator configured to emit optical radiation in a first wavelength range of about 865 nm to about 875 nm, and the other laser oscillator configured to emit radiation in a second wavelength range of about 925 nm to about 935 nm.

23. An optical therapeutic device according to claim 16, wherein said reflector assembly is fixedly coupled to said distal end of said optical fiber.

24. An optical therapeutic device according to claim 16, wherein said reflector assembly is removably coupled to said distal end of said optical fiber.

25. An optical therapeutic device according to claim 16, wherein said reflector assembly has an inner surface having a substantially parabolic cross-section taken along said central axis.

26. An optical therapeutic treatment device according to claim 16, wherein the reflector assembly is configured and arranged for free space forward transmission of secondary blackbody (incandescent) radiation to a target site.

27. An optical therapeutic treatment device according to claim 26, further comprising a lens configured and arranged to receive light from an inner surface of the reflector assembly for free space forward transmission of secondary blackbody (incandescent) radiation to a target site.

28. A kit for treatment of a region of interest in a patient comprising:
A. an optical fiber extending between a proximal end and a distal end, the proximal end configured and arranged to be coupled to an optical energy source for receiving said optical energy incident thereon, wherein said introduced optical energy propagates within said optical fiber to said distal end and exits said optical fiber at said distal end; and
B. a reflector assembly extending along a central axis and coupled to the distal end of the optical fiber, with said distal end positioned within said reflector assembly,
wherein said reflector assembly is adapted to reflect the optical energy emitted from said distal end and propagating radially with respect to said central axis, so that said reflected optical energy propagates at least in part along a propagation axis parallel to said central axis.

29. A kit according to claim 28, further comprising an optical energy source.

30. A kit according to claim 28, wherein said optical energy has a desired degree of coherence ranging from incoherent to coherent.

31. A kit according to claim 28, wherein said distal end of said optical fiber has a carbonized (or capable of being carbonized) distal tip.

32. A kit according to claim 28, wherein said optical energy source is adapted to generate optical energy in a near infrared spectrum range from about 800 nm to about 1100 nm.

33. A kit according to claim 28, wherein said optical energy source includes two laser oscillators, one laser oscillator configured to emit optical radiation in a first wavelength range of about 865 nm to about 875 nm, and the other laser oscillator configured to emit radiation in a second wavelength range of about 925 nm to about 935 nm.

34. An kit according to claim 28, wherein said optical fiber has a fiber cross-section, and said optical therapeutic device further comprising a beam expander axially aligned with said distal end of said optical fiber for receiving optical energy propagating therefrom, and for transmitting said received optical energy with a beam pattern having a greater cross-section than said fiber cross-section.

35. A kit according to claim 34, wherein said beam expander is a Keplerian beam expander.

36. An kit according to claim 34, wherein said beam expander is a Galilean beam expander.

37. An optical therapeutic treatment device according to claim 1, wherein said optical fiber has a fiber cross-section, and said optical therapeutic device further comprising a beam expander axially aligned with said distal end of said optical fiber for receiving optical energy propagating therefrom, and for transmitting said received optical energy with a beam pattern having a greater cross-section than said fiber cross-section.

38. An optical therapeutic treatment device of claim 37, wherein said beam expander is a Keplerian beam expander.

39. An optical therapeutic device according to claim 37, wherein said beam expander is a Galilean beam expander.

40. An optical therapeutic device according to claim 16, wherein said optical fiber has a fiber cross-section, and said optical therapeutic device further comprising a beam expander axially aligned with said distal end of said optical fiber for receiving optical energy propagating therefrom, and for transmitting said received optical energy with a beam pattern having a greater cross-section than said fiber cross-section.

41. An optical therapeutic device according to claim 40, wherein said beam expander is a Keplerian beam expander.

42. An optical therapeutic device according to claim 40, wherein said beam expander is a Galilean beam expander.

43. An optical therapeutic treatment device according to claim 40, wherein the reflector assembly is configured and arranged for free space forward transmission of secondary blackbody (incandescent) NIR radiation to a target site.

44. An optical therapeutic treatment device according to claim 43, further comprising a lens configured and arranged to receive light from an inner surface of the reflector assembly for free space transmission of secondary blackbody (incandescent) radiation to a target site.

45. A method for treating a target infected tissue, prosthetic and/or biofilm in a region of interest in a patient, comprising:
performing a succession of sub-treatments on said region of interest including a first sub-treatment and a second sub-treatment,
wherein said first sub-treatment comprises the steps of:
A1. introducing a tissue and biofilm-penetrating material to said region of interest, said tissue-penetrating material being characterized by optical energy absorption peaks in a range of (incandescent) treatment wavelengths,
A2. providing a first optical fiber having a distal end and a proximal end,
A3. positioning said distal end of said first optical fiber, whereby said distal end is within or adjacent to said region of interest,
A4. at said proximal end, introducing optical energy having a predetermined first energy density and a first spectral range into said first optical fiber whereby said introduced optical energy propagates within said first optical fiber from said proximal end to said distal end, and at said distal end carbonization occurs to incandescently emit optical energy in a second spectral range, wherein said second spectral range is in said range of treatment wavelength, and
wherein said second sub-treatment comprises the steps of:
B1. providing a second optical fiber having a distal end and a proximal end,
B2. positioning said distal end, whereby said distal end is within or adjacent to said region of interest,
B3. at said proximal end of said second optical fiber, introducing optical energy having a second energy density and a third spectral range into said second optical fiber wherein said introduced optical energy propagates within said second optical fiber from said proximal and to said distal end, and at said distal end, exits said second optical fiber, wherein said third spectral range includes wavelengths within an optical energy absorption range of said target infected tissue, prosthetic and or biofilm.

46. The method of claim 45 further comprising:
C. providing an optical therapeutic device, said device including:
a. a housing extending along a central axis;
b. a fiber guide coupled to said housing and adapted to receive an optical fiber having a proximal end and a distal end;
c. a reflector assembly within said housing and extending along said central axis,
wherein said fiber guide is adapted to position the distal end of an optical fiber received therein, to be aligned with said central axis and within said reflector assembly, and
wherein said reflector assembly is adapted to reflect optical energy propagating radially with respect to said central axis, so that said reflected optical energy propagates at least in part along a propagation axis parallel to said central axis;
D. performing said first sub-treatment by positioning said distal end of said first optical fiber to be received within said fiber guide and performing step A4.

47. The method of claim 45 wherein said first sub-treatment is first performed followed by said second sub-treatment.

48. The method of claim 45 wherein said second sub-treatment is first performed followed by said first sub-treatment.

49. The method of claim 45 wherein said tissue-penetrating material is methylene.

50. The method of claim 45 wherein said time integral of said first energy density of said exiting optical energy is greater than or equal to approximately 600-12,000 Joules/cm² and said first spectral range includes wavelengths in the approximate range of 800 nm to 1100 nm.

51. The method of claim 45 wherein said third spectral range includes wavelengths in the approximate range of 870 nm+/−5 nm.

52. The method of claim 45 wherein said third spectral range includes wavelengths in the approximate range of 930 nm+/−5 nm.

53. The method according to claim 52 wherein said third spectral range further includes wavelengths in the approximate range of 870 nm+/−5 nm.

54. The method of claim 45 further comprising the cleaning said region of interest.

55. A method according to claim 45 wherein said target is infected tissue.

56. A method according to claim 45 wherein said target is a prosthetic device.

57. A method according to claim 45 wherein said target is a biofilm.

58. A method for the treatment of a region of interest in a patient comprising:
applying a chromophore dye to the region of interest, the chromophore dye being adapted to absorb light energy comprising at least one absorption peak wavelength in a predetermined range; and
irradiating the region of interest with optical energy generated from an incandescent carbonized distal fiber tip of an optical fiber, wherein said optical fiber having a proximal end and distal end, said proximal end coupled to an optical energy source for receiving optical energy incident thereon, wherein the introduced optical energy propagates within said optical fiber to said carbonized distal tip.

59. The method of claim 58 further comprising the step of cleaning said region of interest.

60. The method of claim 58, wherein said chromophore dye comprises Methylene Blue, Toludine blue, Congo Red, or Malachite Green.

61. The method of claim 58 further comprising
irradiating the region of interest with optical energy in a first wavelength range of about 865 nm to about 875 nm.

62. The method of claim 58 further comprising irradiating the region of interest with optical energy in a second wavelength range of about 925 nm to about 935 nm.

63. The method of claim 59 further comprising irradiating the infected tissue with optical energy in a first wavelength range of about 865 nm to about 875 nm and in a second wavelength range of about 925 nm to about 935 nm.

64. A method according to claim 46, wherein providing a reflector assembly comprises a reflector assembly configured and arranged for free space transmission of Incandescent secondary radiation to a target site.

65. A method according to claim 64, further comprising providing a lens configured and arranged to receive light from an inner surface of the reflector assembly for free space transmission of Incandescent secondary radiation to a target site.

66. A method according to claim 58, further comprising providing a reflector assembly comprises a reflector assembly configured and arranged for free space transmission of incandescent secondary blackbody radiation to a target site.

67. A method according to claim 66, further comprising providing a lens configured and arranged to receive light from an inner surface of the reflector assembly for free space transmission of Incandescent secondary radiation to a target site.

68. An optical therapeutic device according to claim 2, further comprising an energy source and an associated coupling assembly for introducing energy generated by said source to said proximal end of said fiber, wherein said introduced energy generates secondary emission at said distal end of said optical fiber and exits said optical fiber at said distal end.

69. An optical therapeutic device according to claim 68, wherein said energy source is an electrical source.

70. An optical therapeutic device according to claim 69, wherein said energy source is a free electron laser source.

71. A kit according to claim 11, further comprising an energy source and an associated coupling assembly for introducing energy generated by said source to said proximal end of said fiber, whereby said introduced energy generates secondary emission at said distal end of said optical fiber and exits said optical fiber at said distal end.

72. An optical therapeutic device according to claim 71, wherein said energy source is an electrical source.

73. An optical therapeutic device according to claim 72, wherein said energy source is a free electron laser source.

74. A method for treating a target infected tissue, prosthetic and/or biofilm at a target site, comprising:
performing one or more sub-treatments on said target site,
wherein a first sub-treatment comprises:
A1. introducing a tissue-penetrating material to said target site, said tissue-penetrating material being characterized by optical energy absorption peaks in a range of treatment wavelengths;
A2. providing an energy receiving element having a distal end and a proximal end;
A3. positioning said distal end of said energy receiving element, wherein said distal end is within or adjacent to said target site; and
A4. at said proximal end, introducing energy having a predetermined first energy density, wherein said introduced energy propagates within said proximal end to said distal end, and at said distal end carbonization occurs to incandescently emit optical energy in a spectral range in said range of treatment wavelengths.

75. A method according to claim 74, wherein (A4) at said proximal end, introducing energy having a predetermined first energy density, comprises utilizing an source comprising an electrical source.

76. A method according to claim 75, wherein (A4) at said proximal end, introducing energy having a predetermined first energy density, comprises utilizing an source comprising a free electron laser source.

77. A method according to claim 74, further comprising directing the secondary emission through free space to the target site.

78. A method according to claim 74, further comprising irradiating the target site with NIMELs radiation.

79. A method according to claim 78, wherein irradiating the target site with NIMELs radiation occurs prior to (A4) at said proximal end, introducing energy having a predetermined first energy density.

80. A method according to claim 79, wherein irradiating the target site with NIMELs radiation occurs subsequent to (A4) at said proximal end, introducing energy having a predetermined first energy density.
